(19) United States
(12) Patent Application Publication
(10) Pub. No.: US 2018/0030124 A1
(43) Pub. Date: Feb. 1, 2018

(54) METHODS AND COMPOSITIONS FOR ENHANCING ANTI-SSEA4 IMMUNOTHERAPY

(71) Applicant: Lan Bo Chen, Lexington, MA (US)

(72) Inventor: Lan Bo Chen, Lexington, MA (US)

(21) Appl. No.: 15/664,088

(22) Filed: Jul. 31, 2017

Related U.S. Application Data
(60) Provisional application No. 62/368,615, filed on Jul. 29, 2016.

(51) Int. Cl. C07K 16/18; A61K 39/395; A61K 45/06; C07K 16/30

(52) U.S. Cl. CPC C07K 16/18; C07K 16/30; A61K 39/39558; A61K 45/06; C07K 2317/24

(57) ABSTRACT

A method for treating a tumor by administering an antibody or antibody fragment that binds specifically to stage-specific embryonic antigen 4, and an agent that enhances T cell anti-tumor responses. Also provided is a pharmaceutical composition for treating a tumor. The pharmaceutical composition contains an anti-SSEA4 antibody or antibody fragment, an agent that enhances T cell responses, and a pharmaceutically acceptable excipient.

### CROSS-REFERENCE TO RELATED APPLICATIONS

The present application claims priority to Provisional Application No. 62/368,615, filed on Jul. 29, 2016. The content of this prior application is hereby incorporated by reference in its entirety.

### BACKGROUND

The goal of immunotherapy for a tumor is to increase the strength of a patient's own immune responses against the tumor. Immunotherapy can stimulate the activities of specific components of the immune system against tumor cells or can counteract signals produced by the tumor that suppress immune responses.

For example, therapeutic antibodies have been developed that specifically bind to carbohydrate antigens on tumor cells, resulting in death of the cells via recruitment and stimulation of T cells. These carbohydrate antigens, e.g., stage-specific embryonic and stage-specific embryonic antigen 4 (“SSEA4”), are expressed in a wide variety of tumor types and are not expressed in most adult tissues. See, e.g., Lee et al. 2014, J. Am. Chem. Soc. 136:16844-16853. Therapeutic antibodies that specifically bind to SSEA4 are being developed for anti-tumor therapies in view of the tumor-specific expression of this carbohydrate antigen.

The effectiveness of therapeutic antibodies is often limited due to suppression of T cell activity by tumor cells. For example, tumor cells can suppress T cells via activating so-called “immune checkpoint proteins.” This suppression can be counteracted by administering antibodies that block the activity of the immune checkpoint proteins, resulting in an increase in anti-tumor activity of the T cells.

The need exists for anti-tumor immunotherapy methods that overcome the disadvantages of existing treatment modalities.

### SUMMARY

To meet this need, a method is disclosed for treating a tumor by administering to a subject having a tumor an antibody or antibody fragment that binds specifically to stage-specific embryonic antigen 4 (“SSEA4”) and an agent that enhances T cell anti-tumor responses. Cells in the tumor express SSEA4.

Also disclosed is a pharmaceutical composition for treating a tumor. The pharmaceutical composition contains an anti-SSEA4 antibody or antibody fragment, an agent that enhances T cell responses, and a pharmaceutically acceptable excipient.

The details of one or more embodiments of the invention are set forth in the description below. Other features, objects, and advantages of the invention will be apparent from the description and from the claims.

Importantly, all documents cited herein are hereby incorporated by reference in their entirety.

### DETAILED DESCRIPTION

As mentioned above, the method for treating a tumor requires administering an antibody or antibody fragment that binds specifically to SSEA4.

An antibody that specifically binds to SSEA4 can be a chimeric anti-SSEA4 antibody and a fully humanized anti-SSEA4 monoclonal antibody. An antibody fragment that specifically binds to SSEA4 can be, but is not limited to, an anti-SSEA4 Fab and an anti-SSEA4 single chain variable domain. Examples of anti-SSEA4 antibodies and anti-SSEA4 antibody fragments for use in the method of the invention are described in US Patent Application Publication 2016/0102151.

The tumor-treatment method requires administering an agent that enhances T cell anti-tumor responses. As well known in the art, T cell anti-tumor responses can be enhanced via stimulating T cell activation.

Activation of T cells can be stimulated by agents that target co-stimulatory receptors on the cells.

Co-stimulatory receptors include, e.g., tumor necrosis factor receptor superfamily member 4 (“OX40”), glucocorticoid-induced TNFR-related protein (“GITR”), CD137 (“4-1BB”), and CD27.

Specific examples of agents that target co-stimulatory receptors on T cells include, e.g., MEDI6469, MEDI6383, and MOXR0916 (targeting OX40); TRX518 and MK4166 (targeting GITR); urelumab and utolmilumab (targeting 4-1BB); and varlilumab (targeting CD27). See Vilgelm et al. 2016, J. Leukocyte Biol. 100:1-16 (“Vilgelm et al.”) and Hellmann et al. 2016, Advances in Immunol. 130:251-277 (“Hellmann et al.”). One or more of these agents can be administered in the method of the invention for treating a tumor.

On the other hand, T cell anti-tumor responses can be enhanced via blocking T cell inhibitory signals

Blocking T cell inhibitory signals can be accomplished using agents that target T cell inhibitory receptors or their ligands.

Exemplary targets are cytotoxic T lymphocyte-associated protein 4 (“CTLA-4”), programmed cell death-1 (“PD-1”), programmed cell death ligand-1 (“PD-L1”), programmed cell death ligand-2 (“PD-L2”), lymphocyte activation gene-3 (“LAG-3”), T cell immunoglobulin and mucin domain 3 (“TIM-3”), indoleamine 2,3-dioxigenase 1 (“IDO1”), T cell Ig and ITIM domain (“TIGIT”), and B- and T-lymphocyte attenuator (“BTLA”).

Particular examples of agents that target inhibitory receptors on T cells include, e.g., ipilimimab and tremelimumab (targeting CTLA-4); nivolumab, PDR001, MK3475, pembrolizumab, REGN-2810, and pidilizumab (targeting PD-1); BMS-936559, atezolizumab, durvalumab, and avelumab (targeting PD-L1); LAG525 and BMS-986016 (targeting LAG-3); MBG453 (targeting TIM-3); and epacadostat and idoximod (targeting IDO1). See Vilgelm et al. and Hellmann et al.

One or more of the above-identified agents can be administered in the tumor treatment method of the invention. In an embodiment, an agent that stimulates T cell activation and an agent that blocks T cell inhibitory signals are administered together with the anti-SSEA4 antibody or antibody fragment. In one example, the agent that stimulates T cell activation targets OX40 and the agent that blocks T cell inhibitory signals targets PD-1.

Tumors treatable by the above method can be, but are not limited to breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, and brain tumors.

As mentioned above, a pharmaceutical composition of the invention includes an anti-SSEA4 antibody or antibody fragment, an agent that enhances T cell responses, and a pharmaceutically acceptable excipient. The pharmaceutical composition is effective for treating the tumor types mentioned in the preceding paragraph.

The pharmaceutical composition can include any of the anti-SSEA4 antibodies or antibody fragments described above at page 3, paragraph 5, supra.

Agents included in the composition that enhance T cell responses are set forth at page 3, penultimate paragraph through page 4, fourth paragraph, supra. In a particular embodiment, the composition includes two different agents, one that stimulates a T cell co-stimulatory receptor and another that blocks a T cell inhibitory receptor.

The pharmaceutical composition also contains a pharmaceutically acceptable excipient. Examples of such excipients include, but are not limited to, solvents, co-solvents, solubilizers, wetting agents, suspending agents, emulsifiers, chelating agents, antioxidants, reducing agents, antimicrobial preservatives, buffers, bulking agents, protectants, and tonicity adjustors. See e.g., Physician's Desk Reference 71^{st} edition 2016.

Without further elaboration, it is believed that one skilled in the art can, based on the description above, utilize the present invention to its fullest extent.

### Other Embodiments

All of the features disclosed in this specification may be combined in any combination. Each feature disclosed in this specification may be replaced by an alternative feature serving the same, equivalent, or similar purpose. Unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method for treating a tumor, the method comprising administering to a subject having a tumor an antibody or antibody fragment that binds specifically to stage-specific embryonic antigen 4 (SSEA4), and an agent that enhances T cell anti-tumor responses, wherein cells in the tumor express SSEA4.

2. The method of claim 1, wherein the agent that enhances T cell anti-tumor responses is an inhibitor of a target selected from the group consisting of cytotoxic T lymphocyte-associated protein 4 (CTLA-4), programmed cell death-1 (PD-1), programmed cell death ligand-1 (PD-L1), programmed cell death ligand-2 (PD-L2), lymphocyte activation gene-3 (LAG-3), T cell immunoglobulin and mucin domain 3 (TIM-3), indoleamine 2,3-dioxigenase 1 (IDO1), T cell Ig and ITIM domain (TIGIT), B- and T-lymphocyte attenuator (BTLA), and a combination thereof.

3. The method of claim 2, wherein the antibody or antibody fragment is a humanized anti-SSEA4 monoclonal antibody (mAb).

4. The method of claim 3, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

5. The method of claim 1, wherein the agent that enhances T cell anti-tumor responses is a stimulator of a target selected from the group consisting of tumor necrosis factor receptor superfamily member 4 (0X40), glucocorticoid-induced TNFR-related protein (GITR), CD137 (4-IBB), CD27, and a combination thereof.

6. The method of claim 5, wherein the antibody or antibody fragment is a humanized anti-SSEA4 mAb.

7. The method of claim 6, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

8. The method of claim 1, wherein the agent that enhances T cell anti-tumor responses is selected from the group consisting of PDR001, ipilimimab, nivolumab, LAG525, BMS-986016, MBG453,urelumab, utolmilumab, MEDI6469, MEDI6383, varlilumab, tremelimumab, MK3475, MEDI4736, avelumab, durvalumab, pembrolizumab, pidilizumab, epacadostat, idoximod, and a combination thereof.

9. The method of claim 8, wherein the antibody or antibody fragment is a humanized anti-SSEA4 mAb.

10. The method of claim 6, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

11. A pharmaceutical composition for treating a tumor, comprising an anti-SSEA4 antibody or antibody fragment, an agent that enhances T cell responses, and a pharmaceutically acceptable excipient.

12. The pharmaceutical composition of claim 11, wherein the agent that enhances T cell anti-tumor responses is an inhibitor of a target selected from the group consisting of CTLA-4, PD-1, PD-L1, PD-L2, LAG-3, TIM-3, IDO1, TIGIT, BTLA, and a combination thereof.

13. The pharmaceutical composition of claim 12, wherein the anti-SSEA4 antibody or antibody fragment is a humanized anti-SSEA4 mAb.

14. The pharmaceutical composition of claim 13, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

15. The pharmaceutical composition of claim 11, wherein the agent that enhances T cell anti-tumor responses is a stimulator of a target selected from the group consisting of OX40, GITR, 4-1BB, CD27, and a combination thereof.

16. The pharmaceutical composition of claim 15, wherein the anti-SSEA4 antibody or antibody fragment is a humanized anti-SSEA4 mAb.

17. The pharmaceutical composition of claim 16, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

18. The pharmaceutical composition of claim 11, wherein the agent that enhances T cell anti-tumor responses is selected from the group consisting of PDR001, ipilimimab, nivolumab, LAG525, BMS-986016, MBG453,urelumab, utolmilumab, MEDI6469, MEDI6383, varlilumab, tremelimumab, MK3475, MEDI4736, avelumab, durvalumab, pembrolizumab, REGN-2810, pidilizumab, epacadostat, idoximod, and a combination thereof.

19. The pharmaceutical composition of claim 18, wherein the anti-SSEA4 antibody or antibody fragment is a humanized anti-SSEA4 mAb

20. The pharmaceutical composition of claim 19, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.
